(19) United States
(12) Patent Application Publication
(10) Pub. No.: US 2022/0205837 A1
(43) Pub. Date: Jun. 30, 2022

(54) ULTRAVIOLET SENSOR WITH ELECTROCHROMIC INDICATOR

(71) Applicant: L'OREAL, Paris (FR)

(72) Inventors: Richard Besen, New York, NY (US); Haruna Peyret, San Francisco, CA (US); Christopher Hipple, Staten Island, NY (US)

(73) Assignee: L'OREAL, Paris (FR)

(21) Appl. No.: 17/138,753

(22) Filed: Dec. 30, 2020

(51) Int. Cl. G01J 1/02; G01J 1/42; A61B 5/00

(52) U.S. Cl. CPC G01J 1/0219; G01J 1/429; G01J 1/0247; A61B 5/441; G01J 1/0233; A61B 5/0077

(57) ABSTRACT

An electronic detection device with electrochromic indicator is disclosed herein. In one embodiment, the detection device includes a sensor configured to sense a predetermined wavelength, an electrochromic display configured to indicate an intensity of the predetermined wavelength exposure received by the sensor; a capacitor configured for charging by the predetermined wavelength, wherein the capacitor is configured to at least in part power the device; and an antenna configured for communicative coupling with a smart device.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Gathering information about exposure to ultraviolet (UV) light has become increasingly important. For example, individuals may desire information regarding exposure to UV in order to take steps to mitigate the effects of such exposure, including but not limited to avoiding further exposure and using products such as sunscreen that can reduce the harmful effects of exposure. Additionally, with increasing instances of skin cancer and other skin-related afflictions, awareness about skin protection has been increasing. Skin protection can limit or prevent harm to skin from exposure to ultraviolet (UV) electromagnetic radiation. Additionally, it may be beneficial to check a user's exposure to other helpful or harmful wavelengths of light.

As technology progresses, users may want to know their personal exposure to light without checking their cellphone or other smart device. Therefore, systems and methods are needed for improved reporting of personal light exposure readings that also have low power consumption.

In one embodiment, a light detection device includes: a light sensor configured to sense light at a predetermined wavelength; an electrochromic display configured to indicate an intensity of exposure received by the light sensor at the predetermined wavelength; and a capacitor configured for charging by the predetermined wavelength. The capacitor is configured to at least in part power the light detection device. The light detection device also includes an antenna configured for communicative coupling with a smart device.

In one aspect, the predetermined wavelength is an ultraviolet (UV) wavelength.

In one aspect, the electrochromic display is visible to a user. In another aspect, the electrochromic display is powered by the capacitor alone.

In one aspect, the smart device is configured to reset the electrochromic display by communicatively coupling with the antenna of the light detection device.

In one aspect, the electrochromic display comprises at least two electrochromic panels. In another aspect, the electrochromic panels are bi-stable. In yet another aspect, the electrochromic display graphically represents the intensity of the predetermined light exposure in a segmented ring. In one aspect, the individual electrochromic panels comprise electrochromic pixels. In yet another aspect, the electrochromic pixels are activated as the intensity of UV exposure increases.

In one aspect, the smart device is a smart phone. In another aspect, the smart device also includes a rechargeable battery.

In one embodiment, a method of alerting a user about a predetermined wavelength exposure includes: attaching a light detection device to the user; measuring the predetermined wavelength exposure of the user with a light sensor of the light detection device; switching electrochromic pixels from one state to another in response to the predetermined wavelength exposure; and displaying electrochromic ink on an electrochromic display as corresponding to an intensity of user's predetermined wavelength exposure. The electrochromic display is visible to the user. The method also includes resetting the electrochromic display after reaching a maximum predetermined wavelength exposure level.

In one aspect, the predetermined wavelength is an ultraviolet (UV) wavelength.

In one aspect, the method also includes charging a capacitor by the predetermined wavelength exposure.

In one aspect, the method also includes powering the light detection device off the capacitor.

In one aspect, the method also includes: pairing the light detection device to a smart device; and resetting the electrochromic display by communicatively coupling a near field communication (NFC) antenna of the light detection device to the smart device.

In one aspect, the smart device is a smart phone.

In one aspect, the method also includes resetting the electrochromic display after a set time. In another aspect, the method also includes resetting the electrochromic display after 24 hours. In one aspect, the method also includes resetting the electrochromic display after application of a sunscreen.

In one aspect, the method also includes recording a duration of time of user's over-exposure by the smart device. In one aspect, the method also includes setting a threshold of the predetermined wavelength exposure by the user. In yet another aspect, the threshold of predetermined wavelength exposure is determined based on a location where the detection device is attached to the user.

### BRIEF DESCRIPTION OF THE DRAWINGS

The foregoing aspects and many of the attendant advantages of this inventive technology will become more readily appreciated as the same become better understood by reference to the following detailed description, when taken in conjunction with the accompanying drawings, wherein:

FIG. 1 is an example UV exposure detector device in accordance with the present technology;

FIG. 2 is a schematic diagram of example electrochromic pixels in accordance with the present technology;

FIGS. 3A-3F are examples of an electrochromic display exposed to increasing UV intensity in accordance with the present technology;

FIG. 4 is an example interaction between a user and an example UV exposure detector device in accordance with the present technology; and

FIG. 5 is a flowchart of a method of alerting a user to a UV exposure in accordance with the present technology.

### DETAILED DESCRIPTION

While illustrative embodiments have been illustrated and described, it will be appreciated that various changes can be made therein without departing from the spirit and scope of the inventive technology.

In some embodiments, the inventive technology includes an exposure detector device having at least one light sensor capable of sensing a predetermined wavelength. In some embodiments, the light sensor is an ultraviolet (UV) sensor. In other embodiment, the light sensor is a blue light sensor or a light sensor that senses other wavelengths. Therefore, when describing different embodiment in this specification terms “UV sensor” and “light sensor” are used interchangeably.

In some embodiments, the UV sensor also powers the device by charging a capacitor. In some embodiments, the UV exposure detector device transmits data from the UV sensor to a smart device via an NFC antenna. In some embodiments, the UV exposure detector device includes an electrochromic display made up of panels. In some embodiments, these panels are made up of pixels of electrochromic ink. In some embodiments, the electrochromic ink is bi-stable, and can change from one state to another based on intensity of UV light. In some embodiments, the electrochromic display shows a visual of user's UV exposure levels by changing the state of the electrochromic ink as the intensity of UV light increases.

In some embodiments, the NFC antenna is communicatively coupled to a smart device. In some embodiments, tapping the NFC antenna to the smart device reports the user's UV exposure and/or resets the electrochromic display. In some embodiments, the electrochromic display resets after a predetermined amount of time has passed. In some embodiments, the electrochromic display resets after a user applies a countervailing substance, such as a sunscreen.

In some embodiments, the detector device is wearable. In some embodiments, the detector device is carried by a user.

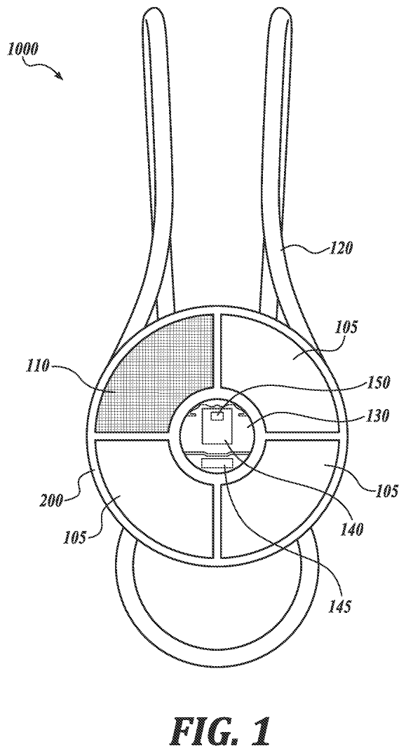

FIG. 1 is an example exposure detector device 1000 in accordance with the present technology. The example exposure detector device 1000 (also referred to as “light detection device” or “light detector device”) includes an attachment 120, a light sensor 130, a capacitor 140, an electrochromic display 200, and an antenna 150. In some embodiments, the electrochromic display 200 is made up of panels 105. In FIG. 1, one of the illustrated panels 105 is activated, displaying visible electrochromic ink 110.

In operation, the light sensor 130 senses light in a predetermined wavelength. For simplicity, the illustrated embodiment includes one light sensor 130, but in other embodiments, the detector device 1000 can have any other number of sensors 130. In some embodiments, the light sensor 130 is a UV sensor or a blue light sensor. The light sensor 130 may be operable at different power consumption levels. The light sensor 130 may be configurable to be deactivated or otherwise placed in a minimal power consumption state when not collecting samples.

In some embodiments, the capacitor 140 is a capacitor charging bank. In operation, when the UV sensor 130 is exposed to UV light, the UV sensor 130 charges the capacitor 140. In some embodiments, UV light sufficiently charges the capacitor 140 such that the capacitor powers entirely on its own the UV exposure detector device 1000. In other embodiments, the UV exposure detector device 1000 (or another light wavelength exposure device) may be battery powered or be powered by a combination of battery 145 and capacitor 140. In some embodiments, the battery 145 may be a rechargeable battery.

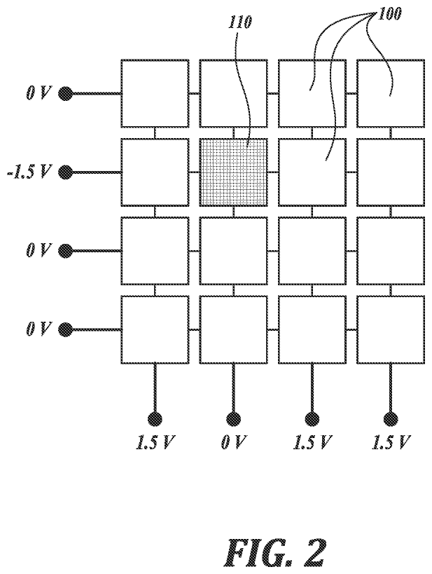

In some embodiments, the electrochromic display 200 shows the user's increasing UV exposure visually with electrochromic ink 110. The electrochromic display 200 includes one or more panels 105. In some embodiments, the panels 105 are made up of one or more pixels 100 (illustrated in FIG. 2). For simplicity, the electrochromic display 200 is illustrated as having four panels 105 in a segmented ring, but in other embodiments, the electrochromic display 200 may include other number of panels 105 in different layout configurations. In some embodiments, the electrochromic display 200 takes other shapes, such as a bar graph.

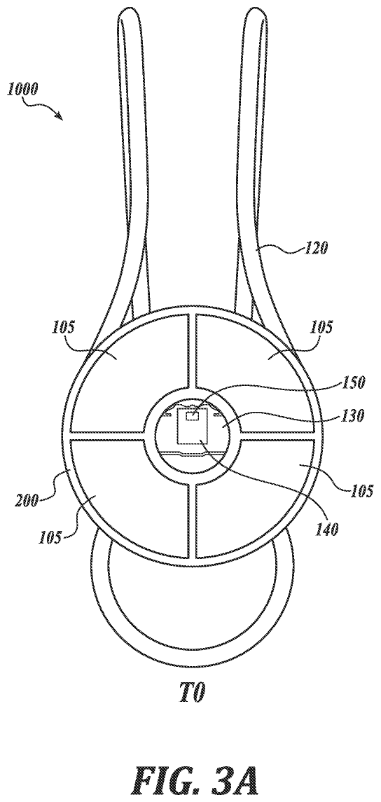

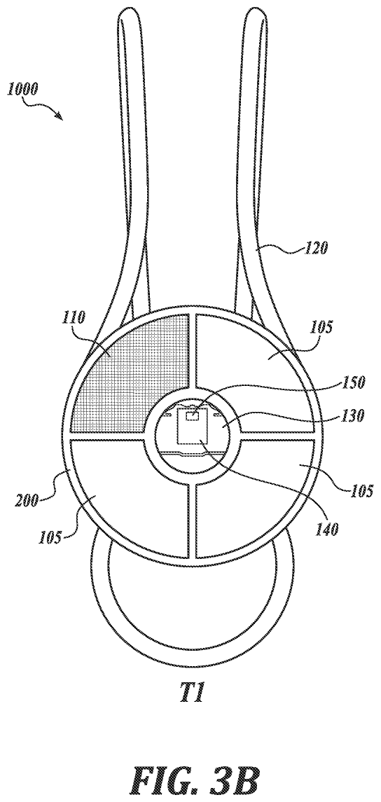

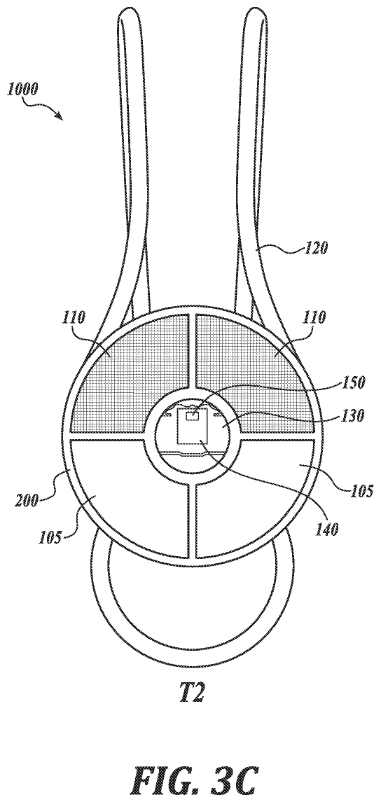

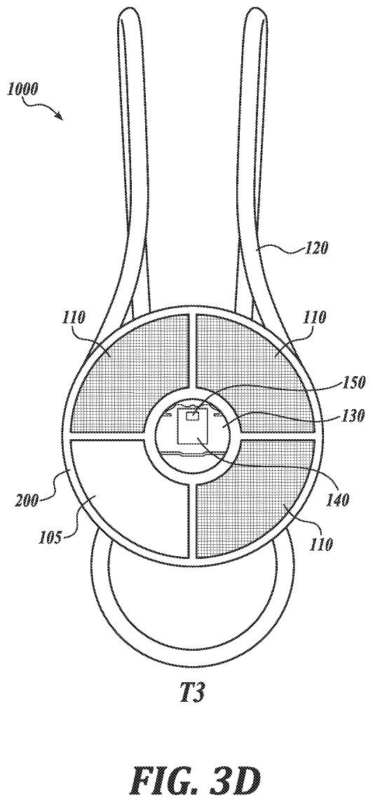

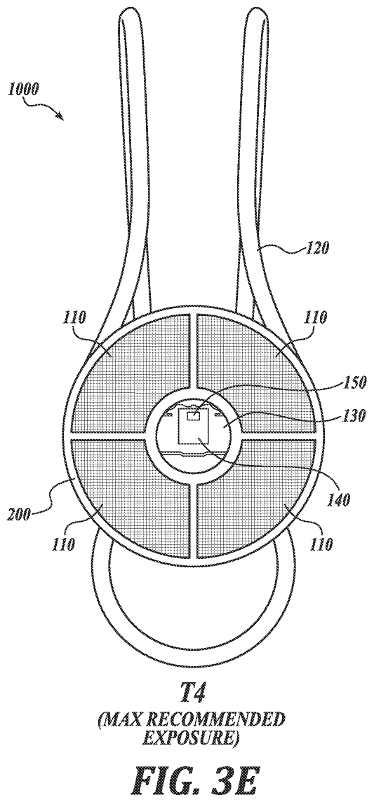

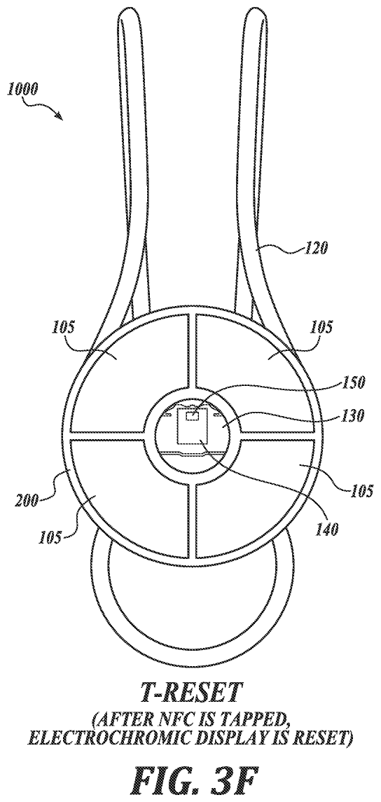

In operation, as the UV sensor 130 senses UV exposure (or exposure to other predetermined wavelengths of light), the electrochromic display 200 shows this exposure visually. As the UV exposure increases, additional panels 105 may be activated to display electrochromic ink 110 (as described in further detail in FIG. 2). For simplicity, a single panel 105 is illustrated as activated, indicating that the user's UV exposure has reached a specific threshold. In some embodiments, the electrochromic display 200 appears blank before being exposed to UV light. In some embodiments, the panels 105 change color as the UV exposure detector device 1000 is exposed to UV light. As the UV exposure increases, more panels 105 may be activated, until the entire electrochromic display 200 is activated (as illustrated in FIG. 3D).

In some embodiments, the antenna 150 is a near field communication (NFC) antenna. In operation, the antenna 150 is communicatively coupled with a smart device (not pictured in FIG. 1). In some embodiments, tapping the NFC antenna 150 to the smart device resets the electrochromic display 200 (as shown in FIGS. 3A-3E). In some embodiments, tapping the NFC antenna 150 to the smart device reports the UV (or other prescribed light wavelength) exposure to the smart device and resets the electrochromic display 200 simultaneously.

FIG. 2 is a schematic diagram of example electrochromic pixels 100 in accordance with the present technology. For simplicity, a series of pixels 100 are illustrated as a pixel array. The pixels 100 are illustrated as arranged into rows and columns but in other embodiments, the pixels 100 may take other configurations. For example, the pixels 100 may be arranged such as to constitute one or more panels 105.

In operation, an electrical impulse (such as voltage, illustrated in FIG. 2) is applied to the pixels 100 by exposure to UV light. In some embodiments, electrochromic ink 110 changes from one state to another. In some embodiments, the electrochromic ink is bi-stable, and can therefore switch back and forth between two states based on combination of voltages applied to the electrochromic ink 110. As explained with reference to FIG. 1 above, the voltages may be entirely or in part provided by the capacitor 140. In some embodiments, the electrochromic ink 110 may change from one state of one color to another state of a different color. In some embodiments, the electrochromic ink 110 may change from a clear state to an opaque state. In such embodiments, the electrochromic ink 110 becomes visible as it is switched into its other state.

As UV light intensity increases, bits within the pixel 100 are flipped, which switches the electrochromic ink 110 from one state to another. For simplicity, a single pixel has been illustrated as switched into a visible electrochromic ink 110 state, but in other embodiments, more than one pixel may be switched at a time. Each pixel 100 or combination of pixels may be programmed to switch into the activated electrochromic ink 110 state at different voltage levels, allowing for some pixels to switch into the visible electrochromic ink 110 state before others. As the voltage from the UV light exposure increases, more and more pixels switch into the activated electrochromic ink 110 state, creating the visual representation on the electrochromic display.

FIGS. 3A-3F are examples of an electrochromic display 200 exposed to increasing UV intensity in accordance with the present technology. The UV exposure detector device 1000 (also referred to as “light detection device” or “light detector device”) includes an attachment 120, an NFC antenna 150, and an electrochromic display 200. For simplicity, the electrochromic display 200 includes four panels 105, but in other embodiments, the electrochromic display 200 may include any number of panels 105. In some embodiments, each panel 105 switches from one electrochromic ink 110 state to another as an equal amount of increased voltage is built up in the electrochromic pixels 100, so that each activated panel represents an equal incremental increase in UV exposure. For simplicity, the electrochromic ink 110 is illustrated as switching from a clear state to an opaque state. In other embodiments, the electrochromic ink may switch from one colored state to another, different colored state.

FIGS. 3A-3F show each stage of the UV exposure detector device 1000 as a user's UV exposure increases. As explained above, the term “UV exposure” encompasses exposure to light at other wavelength (e.g., blue light). For simplicity, six stages (T0, T1-T4 and T-Reset) are illustrated, but in other embodiments, other number of stages can occur to the UV exposure detector device 1000.

In FIG. 3A, the UV exposure detector device 1000 is at stage T0. In some embodiments, T0 occurs when the user attaches the device to themselves or their clothing. Therefore, the UV exposure detector device 1000 has not yet been exposed to UV light at this stage.

In FIG. 3B, the UV exposure detector device 1000 is at stage T1. In T1, exposure to UV light has activated one panel of the electrochromic display 200. As voltage builds in the pixel, the bits within the pixel are flipped, and the electrochromic ink 110 switches from one state to another.

In FIG. 3C, the UV exposure detector device 1000 is at stage T2. The user's exposure to UV light has increased to activate a second panel 105 on the electrochromic display 200. Voltage builds up in the pixel as the duration of UV light exposure increases. The UV light exposure required to flip the second panel 105 into the visible electrochromic ink 110 state is higher than that required to flip the first panel 105.

In FIG. 3D, the UV exposure detector device 1000 is at stage T3. A third panel 105 has been activated by further increasing user's UV exposure.

In FIG. 3E, the UV exposure detector device 1000 is at stage T4. The user's exposure to UV light has activated all panels 105 on the UV exposure detector device 1000. This represents the maximum UV exposure level recommended by the UV exposure detector device 1000 before resetting the electrochromic display 200. In some embodiments, the maximum UV exposure level can be set by the user. In some embodiments, the maximum UV exposure level is hardcoded into the UV exposure detector device 1000. In some embodiments, a smart device (not illustrated in FIGS. 3A-3F) alerts a user when the UV exposure detector device 1000 reaches stage T4. In some embodiments, the smart device recommends that the user applies a countervailing substance such as a sunscreen when the UV exposure detector device 1000 reaches stage T4. In some embodiments, the smart device recommends going inside when the UV exposure detector device 1000 reaches stage T4.

In FIG. 3F, the UV exposure detector device 1000 is at stage T-Reset. In operation, when a user taps the NFC antenna 150 to the smart device, the electrochromic display 200 resets. In some embodiments, the electrochromic display 200 resets after a set amount of time, such as 24 hours. In other embodiments, a user can reset the electrochromic display after applying a countervailing substance such as sunscreen.

For simplicity, stage T-Reset is shown after stage T4, but in some embodiments, the UV exposure detector device 1000 can enter stage T-Reset (and reset the electrochromic display 200) after any stage when the user taps the NFC antenna 150 to the smart device.

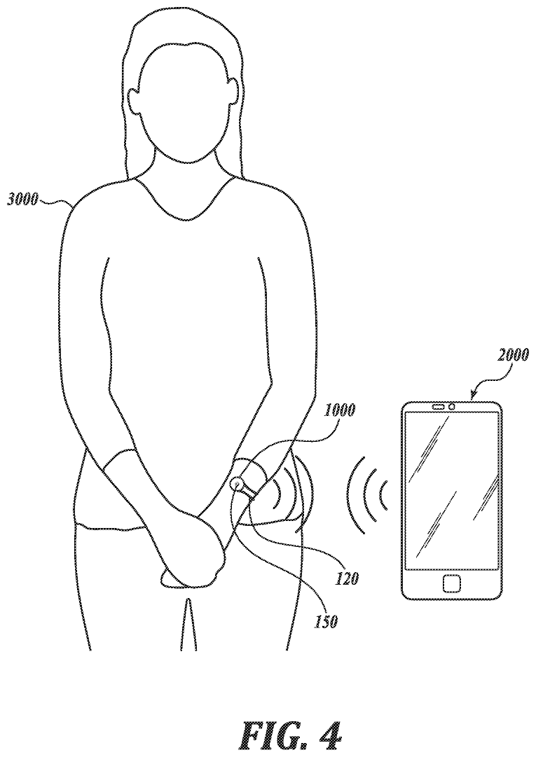

FIG. 4 is an example interaction between a user 3000 and an example UV exposure detector device 1000 in accordance with the present technology. In some embodiments, the UV exposure detector device 1000 is a wearable device. In some embodiments, the UV exposure detector device 1000 includes an attachment 120, for example, a strap that is mounted to a wrist of a user 3000, like a watch. In other embodiments, the detector device 1000 may be mounted to the user's clothing with a clip, a patch or similar attachment 120. In some embodiments, the detector device 1000 is a fob, ID tag, pin, zipper pull, or other form factor that a user 3000 may wear as a necklace or attached to clothing. In some embodiments, the detector device 1000 may be in a form factor designed to be carried rather than worn by the user 3000, such as a case for a mobile phone, or an attachment for a backpack or briefcase.

In operation, the detector device 1000 is in communication with a smart device 2000 via an antenna 150. For simplicity, the smart device 2000 is illustrated as a smart phone, but in other embodiments, the smart device 2000 takes the form of other computing devices such as a smart watch, a tablet, and the like.

In some embodiments, the detector device 1000 is coupled to the smart device 2000 through an NFC antenna 150. The detector device 1000 and the smart device 2000 may communicate using any suitable communication technology, including, but not limited to wireless technologies such as Bluetooth, 2G, 3G, 4G, 5G, LTE, Wi-Fi, WiMAX, and infrared; wired technologies such as USB, Ethernet, FireWire, and Lightning; or combinations thereof. The communication between the detector device 1000 and the smart device 2000 is typically a low-powered communication in order to reduce battery consumption of the smart device 2000, and to allow the UV exposure detector device 1000 to be fully powered by the capacitor (not shown in FIG. 4).

In operation, the UV exposure detector device 1000 senses UV light, and displays a visual representation of the user's UV exposure through an electrochromic display (not shown in FIG. 4). In some embodiments, the exposure detector device 1000 senses a different predetermined wavelength. In some embodiments, the smart device 2000 alerts the user 3000 to their UV exposure at a certain exposure threshold. In some embodiments, the threshold is hardcoded into the smart device 4000 or the detector device 1000. In other embodiments, the threshold is selectable by the user 3000. In some embodiments, the threshold of UV exposure is determined based on a location the UV detection device is attached to the user. The user 3000 may tap the antenna 150 to the smart device 2000 to reset the electrochromic display. In some embodiments, the user 3000 may reset the electrochromic display after any amount of UV exposure. In some embodiments, the user 3000 may reset the electrochromic display after the electrochromic display has reached stage T4 (as shown in FIG. 3D). In some embodiments, the user 3000 may reset the electrochromic display after applying a countervailing substance, such as sunscreen. In some embodiments, the user 3000 may reset the electrochromic display after going inside.

In some embodiments, tapping the antenna 150 to the smart device 2000 reports the user's 3000 UV exposure level to the smart device 2000 instead of, or in addition to, resetting the electrochromic display. In some embodiments, the smart device 2000 may store a duration of time corresponding to the user's 3000 UV over-exposure.

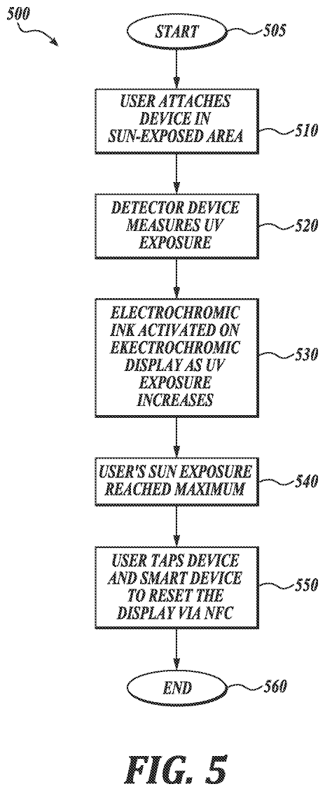

FIG. 5 is a flowchart of a method of alerting a user to a UV exposure in accordance with the present technology. In different embodiments, the method 500 may include additional steps or may be practiced without all steps illustrated in the flow chart.

The method 500 may begin at block 505. In block 510, a user (such as user 3000) attaches a UV detector device (such as UV exposure detector device 1000) to their body in a sun-exposed area. In some embodiments, the sun-exposed area is on the user's wrist (as shown in FIG. 4). In some embodiments, the sun-exposed area is on a user's clothing, such as on a hat or attached to a strap of clothing such as a tank top.

In block 520, the UV exposure detector device measures the UV exposure of the user. In operation, the UV exposure detector device measures UV exposure through one or more UV sensor (such as UV sensor 130). In some embodiments, UV light also powers a capacitor (such as capacitor 140) to power the UV exposure detector device.

In block 530, the electrochromic display (such as electrochromic display 200) begins to display electrochromic ink (such as electrochromic ink 110). As a user's exposure increases, panels (such as panels 105) are activated when pixels (such as pixels 100) accumulate voltage from UV exposure. When the pixels accumulate voltage, the electrochromic ink switches from one state to another, becoming visible to a user, or changing to another color.

In block 540, the user's sun exposure reaches a maximum threshold. In some embodiments, the maximum threshold of UV exposure is hard coded into the UV detector device. In other embodiments, the maximum threshold of UV exposure is set by the user.

In block 550, the user taps the UV detector device to a smart device (such as smart device 2000) to reset the electrochromic display with an NFC antenna (such as antenna 150). In some embodiments, the user resets the electrochromic display after applying or reapplying sunscreen. In some embodiments, the user resets the electrochromic display after going indoors or otherwise removing themselves from UV light exposure. In yet other embodiments, the user resets the electrochromic display after the end of the day. In block 560, the method ends.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. For example, in some embodiments the counter or controller may be based on a low-power buck regulator connected to a capacitor. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” etc., mean plus or minus 5% of the stated value.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure.

What is claimed is:

1. A light detection device comprising:
a light sensor configured to sense light at a predetermined wavelength;
an electrochromic display configured to indicate an intensity of exposure received by the light sensor at the predetermined wavelength;
a capacitor configured for charging by the predetermined wavelength, wherein the capacitor is configured to at least in part power the light detection device; and
an antenna configured for communicative coupling with a smart device.

2. The device of claim 1, wherein the predetermined wavelength is an ultraviolet (UV) wavelength.

3. The device of claim 1, wherein the electrochromic display is visible to a user.

4. The device of claim 1, wherein the electrochromic display is powered by the capacitor alone.

5. The device of claim 1, wherein the smart device is configured to reset the electrochromic display by communicatively coupling with the antenna of the light detection device.

6. The device of claim 1, wherein the electrochromic display comprises at least two electrochromic panels.

7. The device of claim 3, wherein the electrochromic panels are bi-stable.

8. The device of claim 1, wherein the electrochromic display graphically represents the intensity of the predetermined light exposure in a segmented ring.

9. The device of claim 2, wherein individual electrochromic panels comprise electrochromic pixels.

10. The device of claim 8, wherein the electrochromic pixels are activated as the intensity of UV exposure increases.

11. The device of claim 1, wherein the smart device is a smart phone.

12. The device of claim 1, further comprising a rechargeable battery.

13. A method of alerting a user about a predetermined wavelength exposure, the method comprising:
attaching a light detection device to the user;
measuring the predetermined wavelength exposure of the user with a light sensor of the light detection device;
switching electrochromic pixels from one state to another in response to the predetermined wavelength exposure;
displaying electrochromic ink on an electrochromic display as corresponding to an intensity of user's predetermined wavelength exposure, wherein the electrochromic display is visible to the user; and
resetting the electrochromic display after reaching a maximum predetermined wavelength exposure level.

14. The method of claim 13, wherein the predetermined wavelength is an ultraviolet (UV) wavelength.

15. The method of claim 13, further comprising charging a capacitor by the predetermined wavelength exposure.

16. The method of claim 14, further comprising powering the light detection device off the capacitor.

17. The method of claim 13, further comprising:
pairing the light detection device to a smart device; and
resetting the electrochromic display by communicatively coupling a near field communication (NFC) antenna of the light detection device to the smart device.

18. The method of claim 17, wherein the smart device is a smart phone.

19. The method of claim 13, further comprising resetting the electrochromic display after a set time.

20. The method of claim 13, further comprising resetting the electrochromic display after 24 hours.

21. The method of claim 13, further comprising resetting the electrochromic display after application of a sunscreen.

22. The method of claim 17, further comprising recording a duration of time of user's over-exposure by the smart device.

23. The method of claim 13, further comprising setting a threshold of the predetermined wavelength exposure by the user.

24. The method of claim 23, wherein the threshold of predetermined wavelength exposure is determined based on a location where the detection device is attached to the user.
